(19) United States
(12) Patent Application Publication
(10) Pub. No.: US 2023/0360798 A1
(43) Pub. Date: Nov. 9, 2023

(54) ARTIFICIAL INTELLIGENCE (AI) BASED DECISION-SUPPORT DIGITAL ECOSYSTEM TO PROVIDE PERSONALIZED SMART INTEGRATED CARDIO-ONCOLOGY CARE

(71) Applicant: Connecticut Children's Medical Center, Hartford, CT (US)

(72) Inventors: Olga H. Toro-Salazar, Hartford, CT (US); Tze C. Chiam, Hartford, CT (US)

(21) Appl. No.: 18/308,394

(22) Filed: Apr. 27, 2023

Related U.S. Application Data
(60) Provisional application No. 63/335,350, filed on Apr. 27, 2022.

(51) Int. Cl. G16H 50/20; G16H 50/30

(52) U.S. Cl. CPC G16H 50/20; G16H 50/30

(57) ABSTRACT

An Artificial Intelligence (AI) based decision-support system and method to predict cardiotoxicity related outcomes in patients being treated with a cardiotoxic pharmaceutical is provided, wherein the system includes a Data Pipeline (DP) communicated with a Data Repository (DR) and a processing device associated with the data repository. The method includes receiving patient data via the DP, communicating the patient data to the DR and the processing device and processing the patient data to execute at least one of an Artificial Intelligence Algorithm (AIA) and a Machine Learning Algorithm (MLA). The patient data is divided into a training dataset and a validation dataset and the training dataset is processed to generate an AI model to predict a probability of the patient experiencing a cardiac event. The method further includes processing the AI model using the validation dataset to generate an AI Model Accuracy (AIMA) value.

### CROSS-REFERENCE TO RELATED APPLICATIONS

This application claims the benefit of priority of the filing date of U.S. Provisional Patent Application Ser. No. 63/335,350 (Attorney Docket No. VAR0003US) filed on Apr. 27, 2022, the contents of which are incorporated by reference herein in its entirety.

### FIELD OF THE INVENTION

The present invention relates generally to a system for personalizing cardio-oncology care and more particularly to an Artificial Intelligence (AI) based Decision-Support System and Method for personalizing cardio-oncology care.

### BACKGROUND OF THE INVENTION

At any given time, there are approximately 500,000 childhood cancer survivors in the United States. Of these survivors, approximately 300,000 are treated with cardio-toxic anti-cancer therapy and one in ten (10) of these may develop heart failure and die prematurely from heart disease due to the effect on the heart of the cancer medications rather than the cancer itself. One of the main reasons for this is that the doctors don't realize the negative impact the cancer medications have on the heart until it's too late, resulting in a delayed time to intervention. One of the prevailing causes of this involves inefficient workflows the doctors are subjected to due to fragmentation and lack of data/technology and the poor information exchange between all of the parties involved including the provider, family members and the patient.

Current treatment methodology for cardio-oncology care is typically only limited to a few large academic institutions that have highly specialized, resource intensive programs which unfortunately limits access to this type of care. As mentioned above, even with these highly specialized programs, the current standard of cardio-oncology care is fragmented with no adequate coordination among subspecialties. This is disadvantageous and inevitably leads to a delay in the diagnosis of heart damage. And although some data regarding cardio-oncology care is available, typically there is limited access to that data which is typically stored in data silo (EHR, imaging software, consultations in .pdf format, etc.). This is because these data repositories are usually controlled by one department of an organization and may be isolated from other departments of the organization. Unfortunately, this data fragmentation and access isolation results in a manual approach to risk stratification. And although there are a growing number of published national guidelines for cardio-oncology care, these guidelines are unfortunately not incorporated into the daily care of patients. As a result, there is an inability to longitudinally monitor the imaging parameters (heart function data) of the patient, including disease progression, such as heart failure stage and outcome data. Additionally, the lack of quality metrics that are based on modeling of disease outcomes and the limited understanding of the effect of primary and secondary intervention strategies, including cardioprotective therapy and heart failure therapy, disadvantageously results in a reactive treatment approach (i.e., treatment focused) rather than a proactive treatment approach (i.e., prevention focused).

### SUMMARY OF THE INVENTION

An Artificial Intelligence (AI) based decision-support system for providing personalized cardio-oncology care to a patient being treated with a cardiotoxic pharmaceutical is provided and includes a Data Pipeline (DP), a Data Repository (DR) and a processing device associated with the DR. The DP is configured to receive patient data and communicate the patient data to the DR, wherein the DR is configured to receive the patient data from the DP and communicate the patient data to the processing device. The processing device is configured to execute at least one of an Artificial Intelligence Algorithm (AIA) and a Machine Learning Algorithm (MLA) to divide the patient data into a training dataset and a validation dataset, wherein at least one of the AIA and the MLA are configured to process the training dataset to generate an AI model to predict a probability of the patient experiencing a cardiac event. At least one of the AIA and the MLA are configured to process the AI model using the validation dataset to generate an AI Model Accuracy (AIMA) value.

An Artificial Intelligence (AI) based decision-support system for providing personalized cardio-oncology care to a patient being treated with a cardiotoxic pharmaceutical is provided and includes a Data Pipeline (DP), a Data Repository (DR), and a processing device associated with the DR, wherein the DP is configured to receive patient data and communicate the patient data to the DR. The DR is configured to receive the patient data from the DP and communicate the patient data to the processing device, wherein the processing device is configured to execute at least one of an Artificial Intelligence Algorithm (AIA) and a Machine Learning Algorithm (MLA) to process at least a portion of the patient data to generate an AI model to predict a probability of the patient experiencing a cardiac event and to generate and AI Model Accuracy (AIMA) value.

A method for training an Artificial Intelligence (AI) based decision-support system to predict cardiac outcomes in patients being treated with a cardiotoxic pharmaceutical is provided, wherein the system includes a Data Pipeline (DP) communicated with a Data Repository (DR) and a processing device associated with the data repository. The method includes receiving patient data via the DP, communicating the patient data to the DR and the processing device and processing the patient data to execute at least one of an Artificial Intelligence Algorithm (AIA) and a Machine Learning Algorithm (MLA) to divide the patient data into a training dataset and a validation dataset. The method further includes processing the training dataset to generate an AI model to predict a probability of the patient experiencing a cardiac event, and processing the AI model using the validation dataset to generate an AI Model Accuracy (AIMA) value.

### BRIEF DESCRIPTION OF THE DRAWINGS

The foregoing and other features and advantages of the present invention should be more fully understood from the accompanying detailed description of illustrative embodiments taken in conjunction with the following Figures in which like elements are numbered alike in the several Figures:

FIG. 1A is a high-level flow diagram of an Artificial Intelligence (AI) based Decision-Support System and Method (DSSM), in accordance with an embodiment of the invention;

FIG. 1B is a high-level flow diagram of the technology infrastructure that may support the Artificial Intelligence (AI) based Decision-Support System and Method (DSSM) of FIG. 1A, in accordance with an embodiment of the invention;

FIG. 1C is a high-level flow diagram of the data systems that may support the Artificial Intelligence (AI) based Decision-Support System and Method (DSSM) of FIG. 1A, in accordance with an embodiment of the invention;

FIG. 2 is a high-level flow diagram of the Artificial Intelligence (AI) based Decision-Support System and Method (DSSM) for FIG. 1A, in accordance with an embodiment of the invention;

FIG. 3 is a high-level flow diagram of the clinical and technology workflow of the Artificial Intelligence (AI) based Decision-Support System and Method (DSSM) of FIG. 1A, in accordance with an embodiment of the invention;

FIG. 4 is a high-level flow diagram of a Cloud Based Decision Support Platform (CDBSP) of the Artificial Intelligence (AI) based Decision-Support System and Method (DSSM) of FIG. 1A, in accordance with an embodiment of the invention;

FIG. 5 is a high-level flow diagram of a patient accessible mobile app of the Artificial Intelligence (AI) based Decision-Support System and Method (DSSM) of FIG. 1A, in accordance with an embodiment of the invention;

FIG. 6 is a high-level operational flow block diagram illustrating a method for implementing an Artificial Intelligence (AI) based Decision-Support System, in accordance with an embodiment of the invention;

FIG. 7 is a block diagram illustrating cancer treatment stages, in accordance with an embodiment;

FIG. 8 is a scoring sheet illustrating scoring methodology for use with the Artificial Intelligence (AI) based Decision-Support System of FIG. 1A, in accordance with an embodiment;

FIG. 9 is a risk stratification guideline sheet illustrating clinal care treatment pathways for use with the Artificial Intelligence (AI) based Decision-Support System of FIG. 1A, in accordance with an embodiment;

FIG. 10 is an operational flow block diagram illustrating clinal care treatment actions for use with the Artificial Intelligence (AI) based Decision-Support System of FIG. 1A, in accordance with an embodiment;

FIG. 11A is an operational flow block diagram illustrating clinal care treatment actions for use with the Artificial Intelligence (AI) based Decision-Support System of FIG. 1A, in accordance with an embodiment;

FIG. 11B is an operational flow block diagram illustrating a clinal care protocol for use with the Artificial Intelligence (AI) based Decision-Support System of FIG. 1A in treating APML with arsenic, in accordance with an embodiment;

FIG. 11C is a block diagram illustrating a list of cardiotoxic agents and effects for use with clinal care treatment actions for use with the Artificial Intelligence (AI) based Decision-Support System of FIG. 1A, in accordance with an embodiment;

FIG. 12A is an operational flow block diagram illustrating an ECHO algorithm for use with the Artificial Intelligence (AI) based Decision-Support System of FIG. 1A, in accordance with an embodiment;

FIG. 12B is an operational flow block diagram illustrating an ECHO algorithm for use with the Artificial Intelligence (AI) based Decision-Support System of FIG. 1A, in accordance with an embodiment;

FIG. 13 is an operational flow block diagram illustrating an MRI algorithm for use with the Artificial Intelligence (AI) based Decision-Support System of FIG. 1A, in accordance with an embodiment; and

FIG. 14A is a dosing regimen for Dexrazoxane for the Artificial Intelligence (AI) and Machine Language (ML) model for use with the Decision-Support System of FIG. 1A, in accordance with an embodiment.

FIG. 14B is a dosing regimen for Administration of Carvedilol for the Artificial Intelligence (AI) and Machine Language (ML) model for use with the Decision-Support System of FIG. 1A, in accordance with an embodiment.

FIG. 14C is an operational flow block diagram illustrating an endocrinological treatment protocol for the Artificial Intelligence (AI) and Machine Language (ML) model for use with the Decision-Support System of FIG. 1A, in accordance with an embodiment.

FIG. 15 is an operational flow block diagram illustrating the Artificial Intelligence (AI) and Machine Language (ML) model for use with the Decision-Support System of FIG. 1A, in accordance with an embodiment.

FIG. 16 is an operational flow block diagram illustrating a development workflow for Artificial Intelligence (AI) and Machine Language (ML) model for use with the Decision-Support System of FIG. 1A, in accordance with an embodiment.

### DETAILED DESCRIPTION

It should be appreciated that in an embodiment, the present invention may include a platform that uses a data repository configured for integration of real-world data arising from clinical practice for the development of machine learning models for predicting outcomes. The platform may be a cloud-based platform that is configured to optimize and personalize risk adapted cardio-oncology care to individual pediatric patients, starting from diagnosis and continuing throughout every stage of the patient's treatment. A mobile platform allows a patient the ability to have their care personalized with coaching and monitoring by professionals, including integration of patient reported outcomes and exercise intervention to mitigate cardiotoxicity and outcomes associated with cardiotoxicity. Date reported outcomes including physical activity and cardiac symptoms obtained through the application may contribute to the continuous stream of data being introduced to the data repository.

It should be appreciated that in an embodiment, the foundational basis of the platform may be a cloud-based data repository for the creation of algorithms that will predict outcomes related to cardiotoxicity. This combined cloud-based decision support platform and mobile app may allow for the early identification of patients based on cancer diagnosis and exposure to cardio toxic cancer therapy. The platform of the invention may provide precision-based care through risk stratification into low risk, intermediate risk and high risk categories of heart failure and, may assign primary and secondary prevention strategies through clinical pathways of care based on the risk level. It should be further appreciated that risk stratification may start at the time of diagnosis and may continue to be implemented throughout the patient's care at multiple time points during the patient's care. This continuous data collection may allow the patient care to be modified based, at least in part, on changes in the patient's risk level. It should be appreciated that this unique and novel Artificial Intelligence (AI) based Decision-Support System and Method (DSSM) may significantly reduce the time to intervention by 1) identifying the patients that are at risk earlier than current practices allow, 2) defining and implementing a patient's personalized path of treatment faster than current practices allow, and 3) tracking, in real time, all relevant data to ensure a timely intervention and more personalized treatment regimen. Optimizing the workflow with these elements will advantageously lead to more favorable treatment outcomes and reduced mortality.

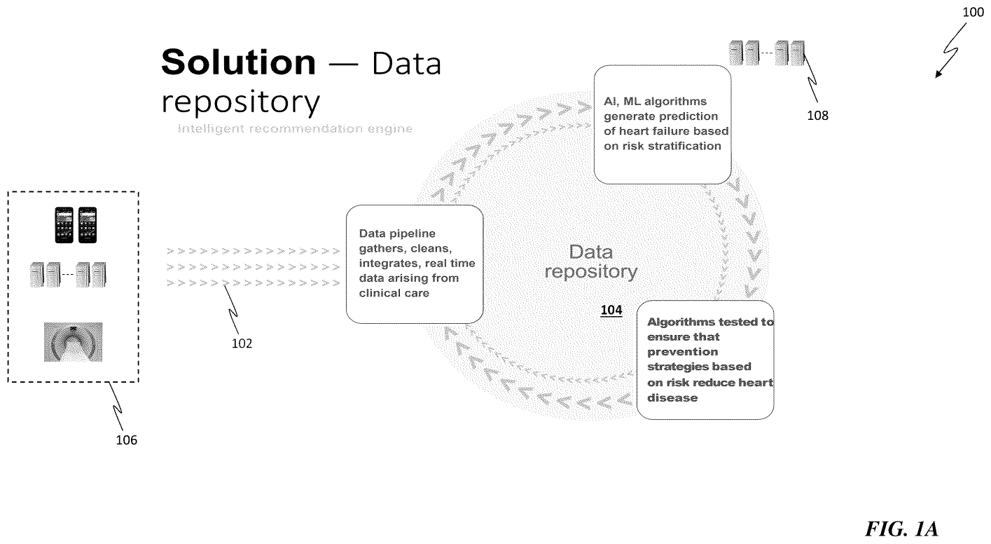

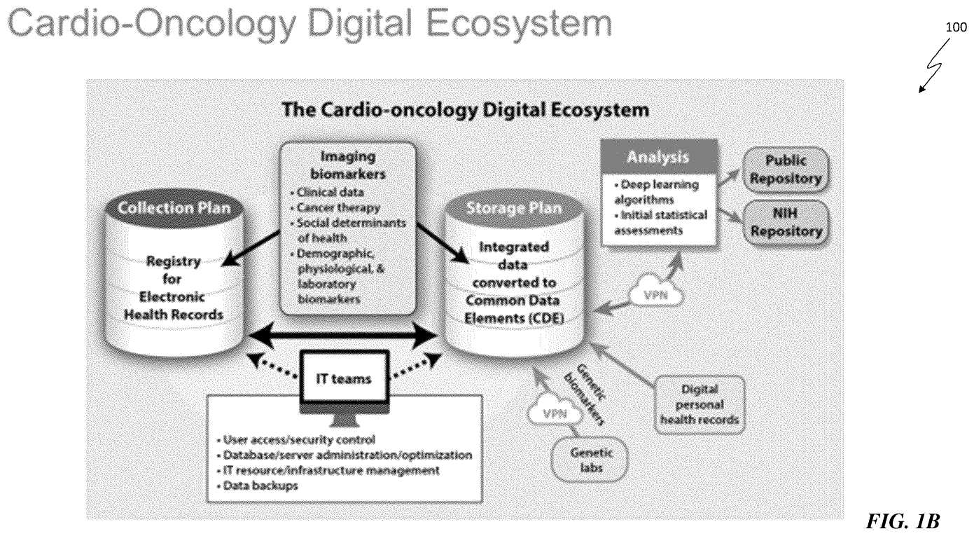

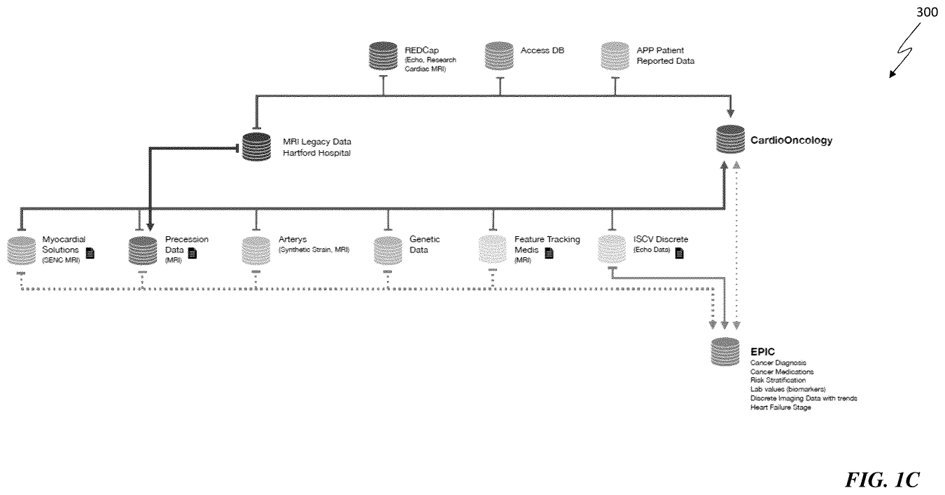

Referring to the Figures, an embodiment of the invention is presented and implemented via a multi-part solution approach. Referring to FIG. 1A, FIG. 1B and FIG. 1C, a high-level operational block diagram illustrating a general concept of an Artificial Intelligence (AI) based Decision-Support System (i.e., Cardio-oncology Digital Ecosystem) and Method (DSSM) is shown, in accordance with an embodiment. The AI-DSSM may be configured to encourage a health care provider to investigate the possible exposures to cardiotoxic cancer therapies that a patient that has been diagnosed with cancer will experience. This will aid in the early diagnosis of any possible heart related issues, such as heart failure and/or heart disease. The health care provider may perform a stratification exercise to determine the patient's level of risk of heart failure and/or heart disease that the patient may experience due to the exposure to cardiotoxic cancer therapy. In response, the health care provided may develop personalized, appropriate and precise prevention strategies and clinical pathways for the patient based on the patient's individual risk. This approach may be implemented throughout every stage of the patient's treatment to minimize the patient's risk of cardiac issues, such as heart failure and/or heart disease.

In accordance with an embodiment, a system for predicting cardiotoxicity of a patient being treated for cancer is provided and includes an Intelligent Recommendation Engine (IRE) 100 having a Data Pipeline (DP) 102 communicated with a Data Repository (DR) 104. The DP 102 may include one or more computer servers and/or processing devices that are configured to receive patient data and communicate the patient data to the DR 104. Moreover, the DP 102 may be communicated with the DR 104 via hardwired and/or wireless communications. The DP 102 may include and/or may be communicated with one or more DP Input Devices (DPID) 106 which may be configured to obtain patient data (i.e., ECG/EKG machine, a blood pressure machine, a heart rate monitor, an MRI machine, a CT Scanner, an ultrasound machine, etc.) and/or allow for patient data to be entered (i.e., Computer, Laptop, Tablet, Smartphone, PDA, Patient Records Registry, etc.). The DR 104 may be associated with one or more processing devices 108 which may be configured to process data received from the DP 102 responsive to one or more algorithms, such as AI/ML algorithms, wherein one or more of the one or more processing devices 108 may be located remotely to the DR 104 and/or they may be part of the IRE 100.

Furthermore, it is contemplated that, in an embodiment, the DP 102 and or DR 104 may be accessible by a variety of health care providers/networks to allow and encourage submission of patient data from a variety of sources throughout the country and the world. Accordingly, the IRE 100 generally operates by receiving patient data into the DP 102 via one or more DPIDs 106. The patient data is then introduced into the DR 104, where the patient data is introduced to the one or more processing devices 108 which process the patient data responsive to one or more AI/ML algorithms to generate predictive analysis which may predict the probability that a patient will experience cardiotoxicity and suggest treatment/prevention strategies. It should be appreciated that while this processing is being performed, additional data will be introduced into the DP 102 and hence, the DR 104 and the one or more processing devices 108. The predicted data will 1) be reintroduced into the one or more processing devices 108 to update the patient data and predictive information, and 2) processed to try and ensure the integrity of the predictions and treatment/prevention strategies. With every introduction iteration of new data and old data, the predictions and treatment/prevention strategies may become more accurate and robust. Moreover, it is contemplated that in some embodiments, the patient may be allowed to enter data into the IRE 100. Accordingly, patient data is continuously being introduced into the system (and the algorithms) to allow the IRE 100 to learn from the continuous stream of data being introduced therein.

Generally, the AI-DSSM involves the implementation of a plurality of solution approaches. The first part of the solution approach involves the use of an Intelligent Recommendation Engine (IRE) 100 which includes a Data Pipeline (DP) 102 and a Data Repository (DR) 104. The second part of the solution approach involves the use of Cloud Based Decision Support Platform (CBDSP) 200 which is configured to allow a provider to be involved and/or control some or all of the aspects of the solution approach and the third part of the solution approach which involves a Mobile Application (MA) 300 to allows the patient to be directly involved in the solution approach.

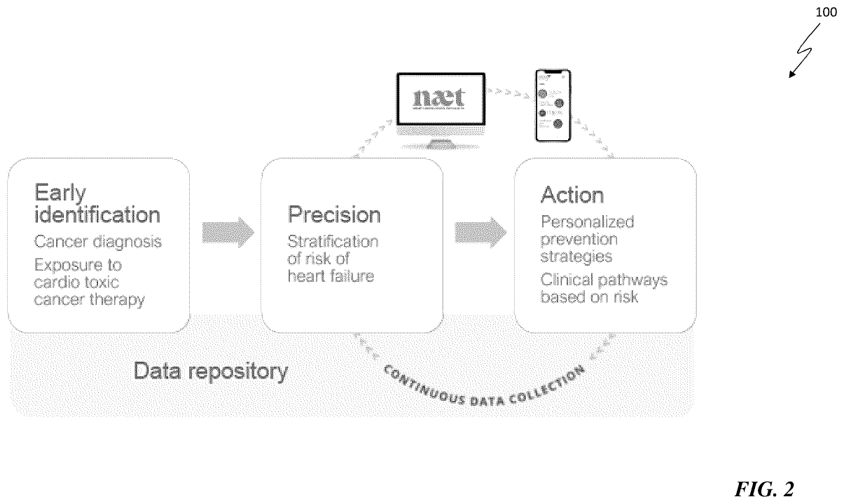

With regards to a first part of the solution approach and referring to FIG. 1A, FIG. 1B, FIG. 1C and FIG. 2, a high-level operational flow block diagram illustrating the data flow within the Intelligent Recommendation Engine (IRE) 100 is shown, in accordance with one embodiment. As shown, real-time patient data which may be obtained by a health care provided from clinical care of the patient. This real-time patient data is introduced into the DP 102, wherein the DP 102 generates processed patient data by gathering, cleaning and integrating the real-time patient data that is introduced into the DP 102. This processed patient data is introduced into the DR 104 which may then introduce the processed patient data to one or more Artificial Intelligence (AI) and/or Machine Language (ML) algorithms. It should be appreciated that the AI and/or ML algorithms may be resident on one or more processing devices and may be configured to generate prediction data responsive to whether the patient will likely experience heart failure and/or other heart related issues, such as heart disease, wherein the prediction data is based, at least in part, on the patient's risk level. The AI and/or ML algorithms may then process the prediction data to recommend prevention and risk reduction strategies that the patient may undertake to prevent heart related issues and/or reduce his/her risk level of experiencing heart disease and/or other heart related issues.

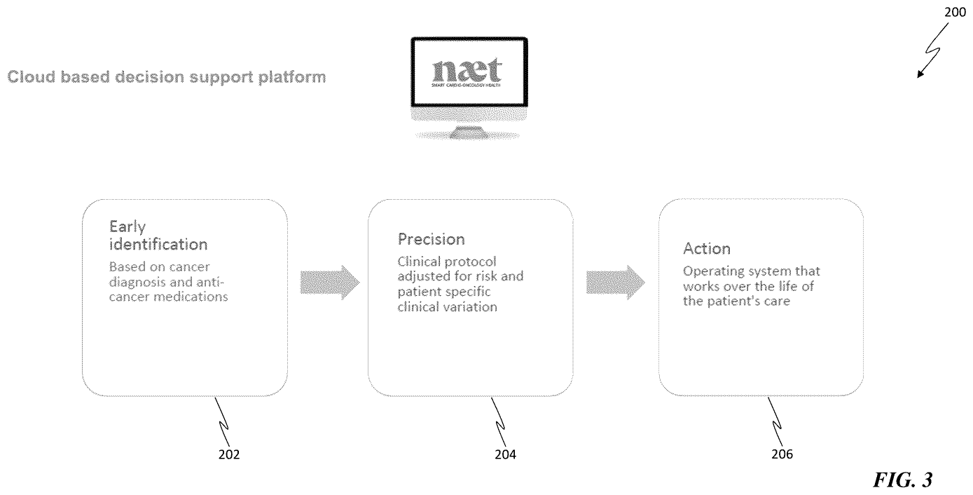

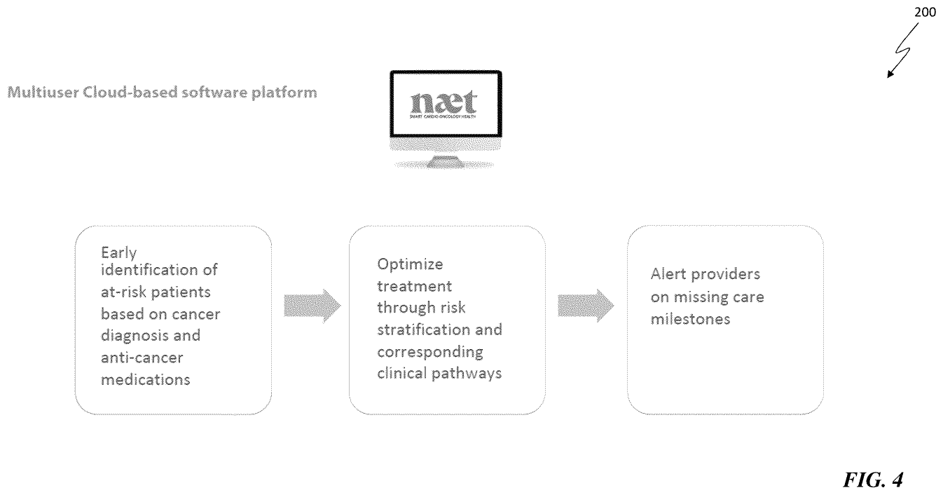

With regards to a second part of the solution approach and referring to FIG. 3 and FIG. 4, a high-level operational block diagram illustrating the CBDSP 200 is shown, in accordance with an embodiment. The second part of the solution approach involves the provider implementing the CBDSP 200 to implement an early identification step 202, where the provider acts to recognize cardiotoxicity, and/or the possibility of cardiotoxicity, based on the cancer diagnosis of the patient, associated cardiovascular comorbidities, the anti-cancer medications that will be used to treat the patient and the genetic predisposition of the patient. The second part of the solution further involves the provider assessing the risk level of the patient and adjusting the clinical protocol responsive 204 to the assessed risk level of the patient and/or a patient specific clinical variation. CBDSP 200 may then be implemented on a continuous basis 206 over the life of the patient's care.

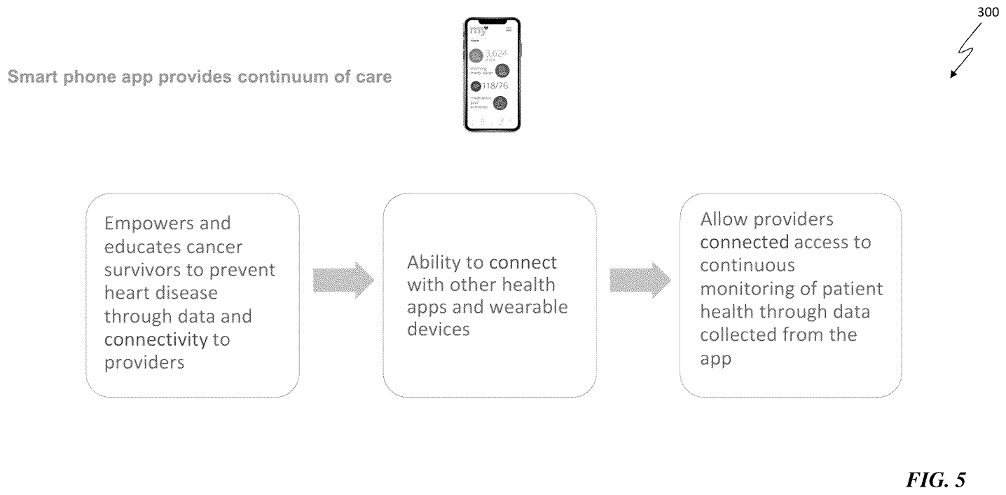

With regards to a third part of the solution approach and referring to FIG. 5, a high-level operational block diagram illustrating the patient-based MA 300 is shown, in accordance with an embodiment. The MA 300 may be configured to empower and educate cancer patients and/or survivors on how to prevent heart disease through data and connectivity to health care providers. The MA 300 may be configured to allow a patient to connect with other health related applications and wearable devices. Moreover, the MA 300 may be configured to obtain data from the patient to allow health care providers to have connected access to a patient and to establish a continuous monitoring capability/relationship with the patient to allow the health care provider to continuously be aware of the health of the patient. It should be appreciated that the MA 300 may be implemented via any device that is capable of wireless and/or wired communication, such as a computer, iPod, iPad, MP3 Player, a PDA, a Pocket PC and/or a Cell phone with wireless and/or wired connection capability. This advantageously allows for a continuum of patient care throughout the patient's cancer treatment.

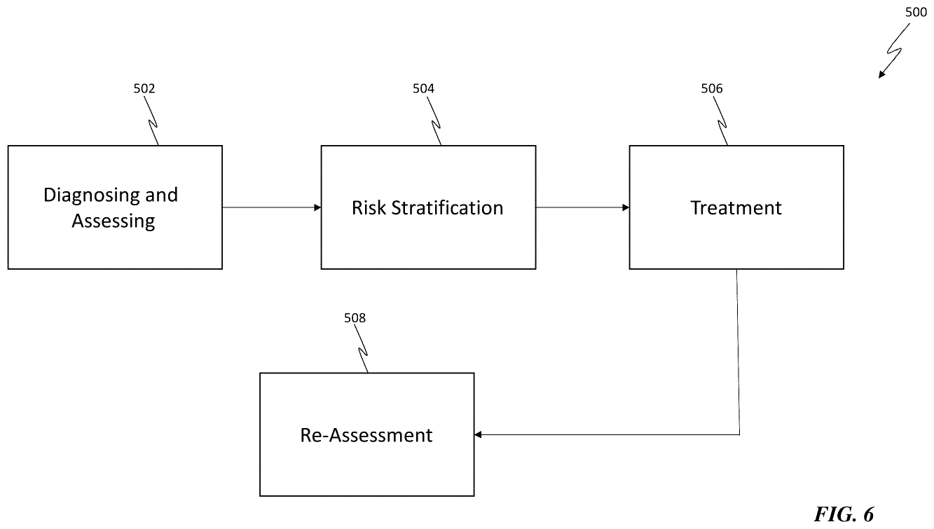

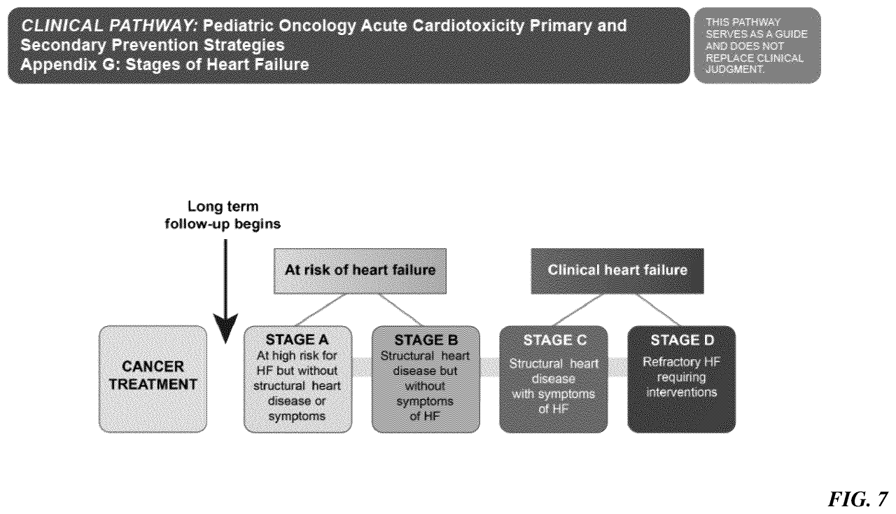

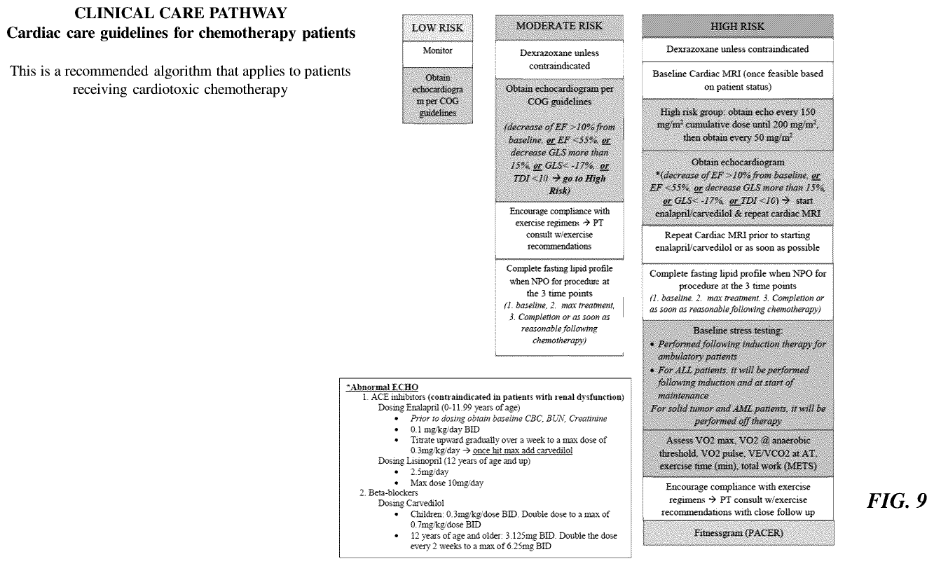

Referring to FIG. 6-FIG. 14, a high-level operational flow block diagram illustrating a method 500 for implementing an Artificial Intelligence (AI) based Decision-Support System 600 is shown, in accordance with one embodiment of the invention. As shown in FIG. 6, the method 500 includes diagnosing and assessing the patient, as shown in operational block 502. Diagnosing and assessing the patient includes performing an evaluation of the patient for multiple characteristics. This evaluation includes a health care provider assessing social determinants of the patient's health and the physical fitness of the patient. The health care provider may also conduct a nutrition evaluation of the patient's eating habits and/or perform quality of life assessment of the patient. The health care worker may obtain a baseline echocardiogram per COG protocols, a baseline cardiac MRI (if indicated), baseline labs as ordered by Cardiology (i.e., troponin I (Tnl), N-terminal pro-BNP (NT-proBNP), vitamin D, etc.), baseline labs as ordered by Heme-Oncology (i.e., lipid panel (may include triglycerides), fructosamine, HbA1C, ferritin, etc.) and, if possible, consent for collection of a blood sample for future biomarker and genetic research.

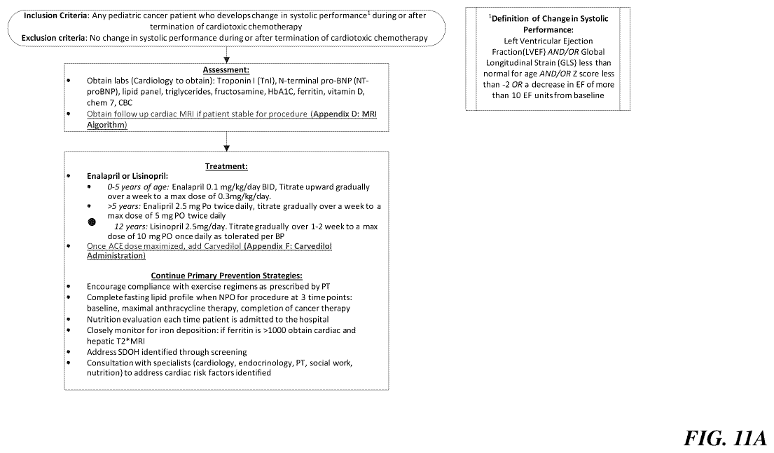

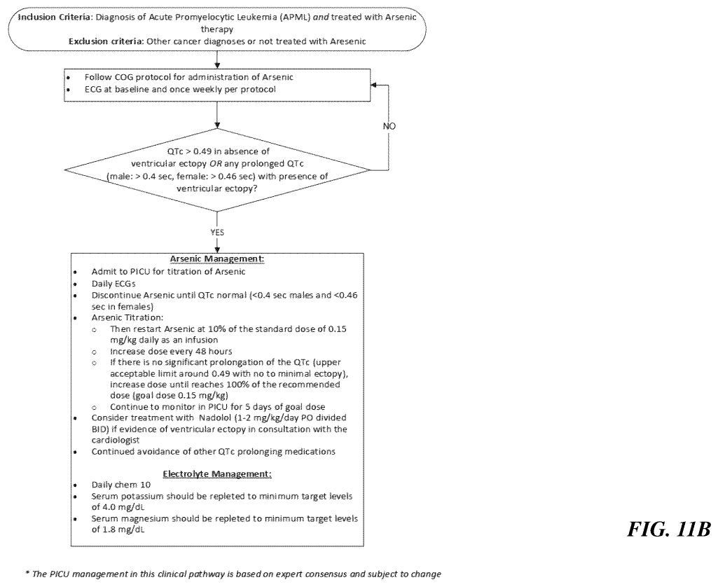

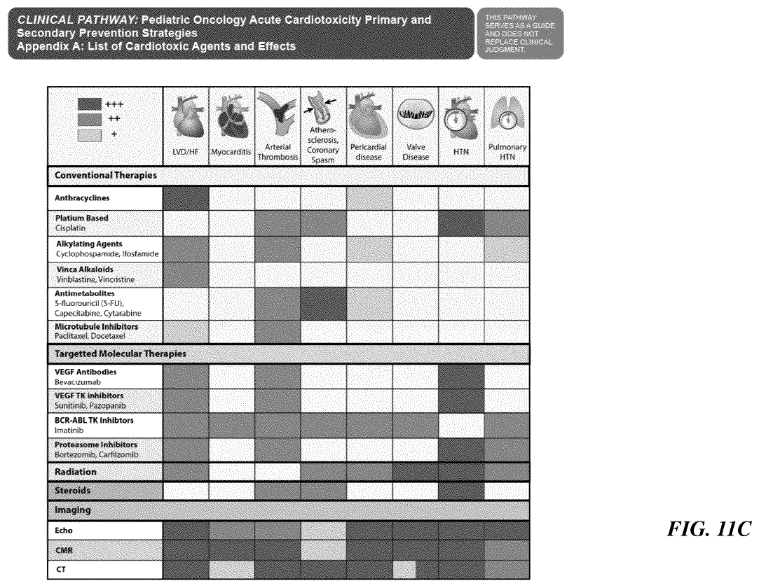

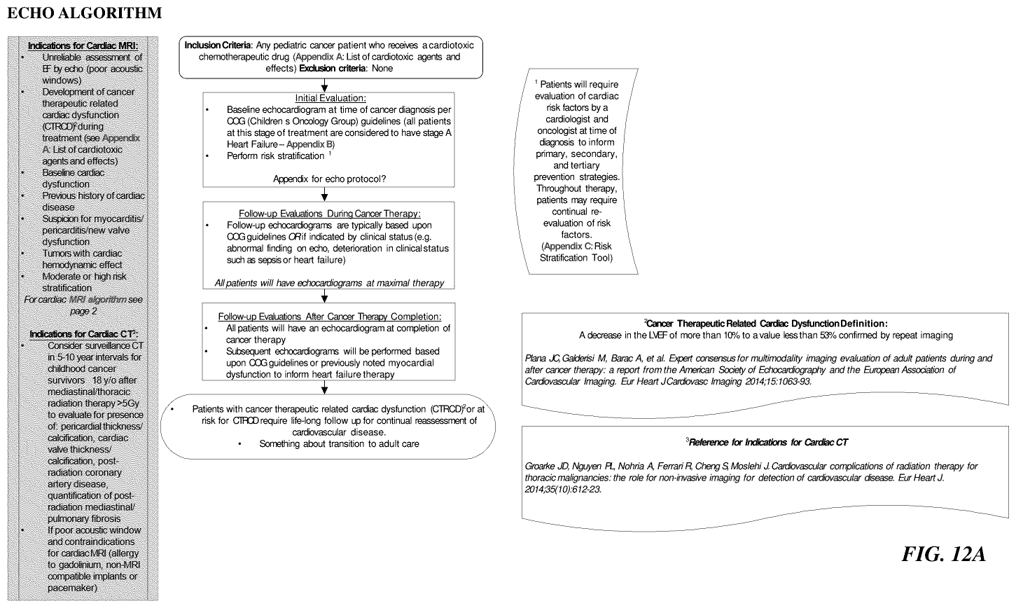

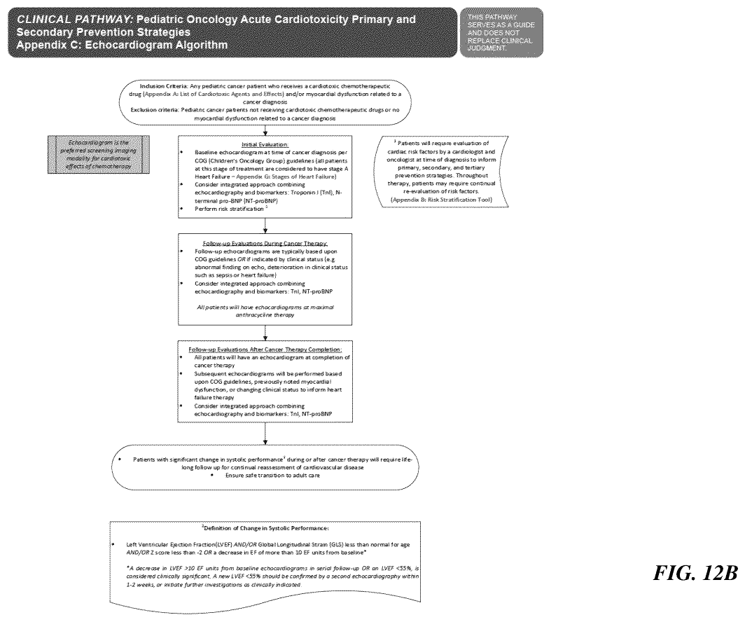

Referring to FIG. 11A, FIG. 11B and FIG. 11C, one of treatment criteria is illustrated and includes Arsenic Management and a list of some of the cardiotoxic agents and effects. It should be appreciated that if the health care worker desires to have an echocardiogram performed, one embodiment of an echocardiogram algorithm that may be followed is shown in FIG. 12A and FIG. 12B.

The health care provider may also conduct and/or implement a Primary Prevention Strategy (PPS) for the patient. This health care provider may encourage compliance by the patient to 1) an exercise regime as may prescribed by PT (i.e., Reference: Pediatric Oncology Exercise Manual), 2) complete a fasting lipid profile when NPO for procedure at 3 time points (baseline, maximal anthracycline therapy, and completion of chemotherapy), 3) Nutrition evaluation each time the patient is admitted to the hospital, 4) Closely monitor for iron deposition (if ferritin is >1000 ng/ml the health care provider may obtain a cardiac and hepatic T2*MRI), 5) address social determinants of health that were identified during the assessment and/or through screening, and 6) consultation with specialists (i.e., cardiology, endocrinology, PT, social work, nutrition, etc.) to address cardiac risk factors that were identified. It should be appreciated that a baseline cardiac MRI may be indicated if one or more of the following is present, 1) there is an unreliable assessment of EF by echo (poor acoustic windows), 2) there is development of cancer therapeutic related cardiac dysfunction (CTRCD) during treatment, 3) there is baseline cardiac dysfunction, 4) there is previous history of cardiac disease, 5) there is suspicion that the patient has myocarditis/pericarditis/new valve dysfunction, 6) there is one or more tumors with cardia hemodynamic effect and/or 7) there is a moderate or high risk stratification assessment.

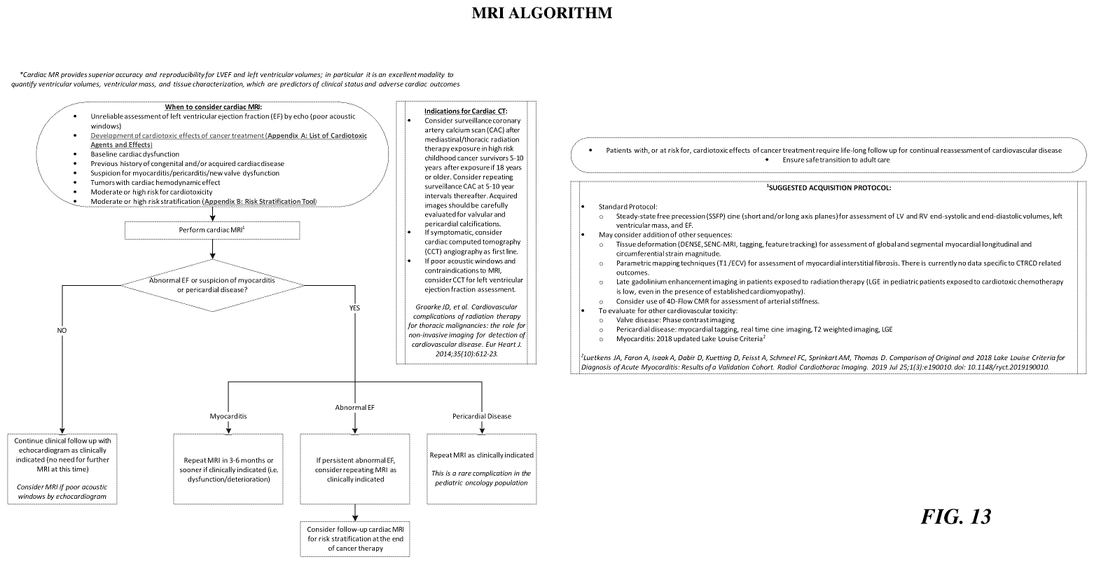

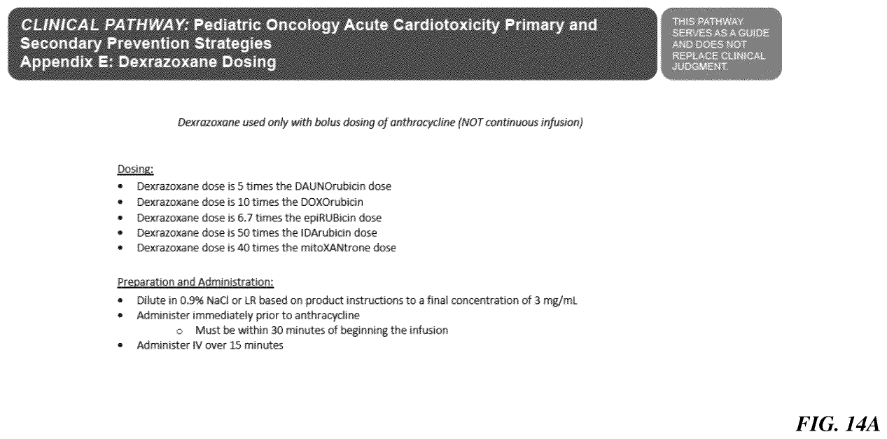

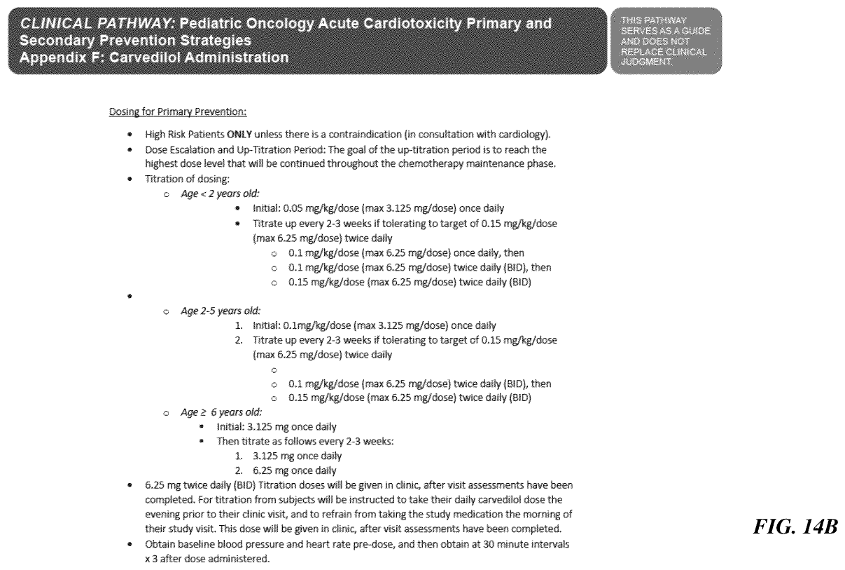

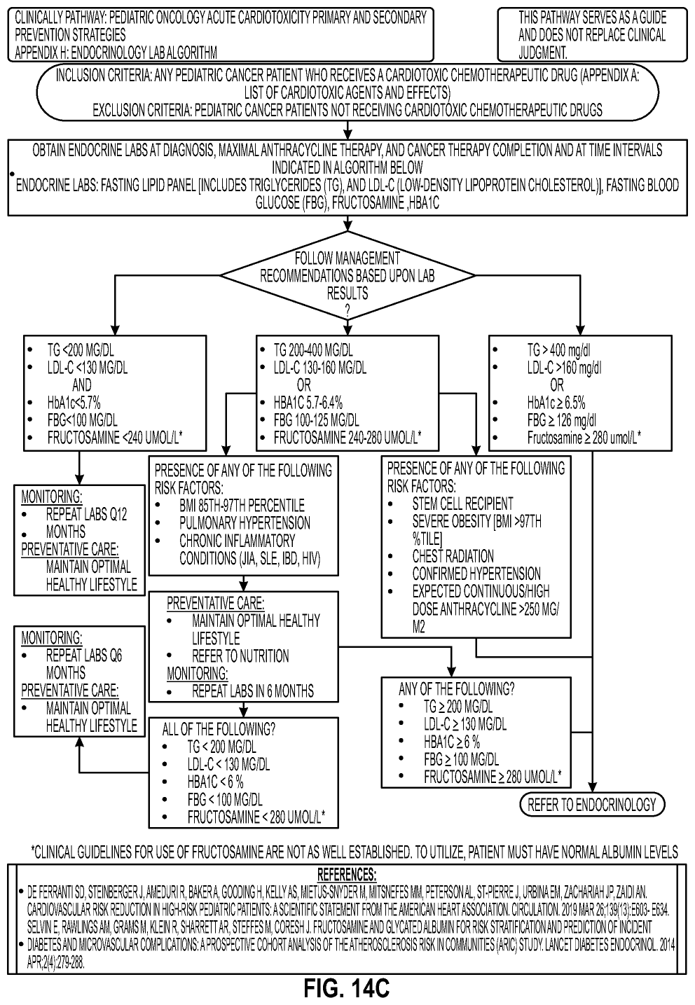

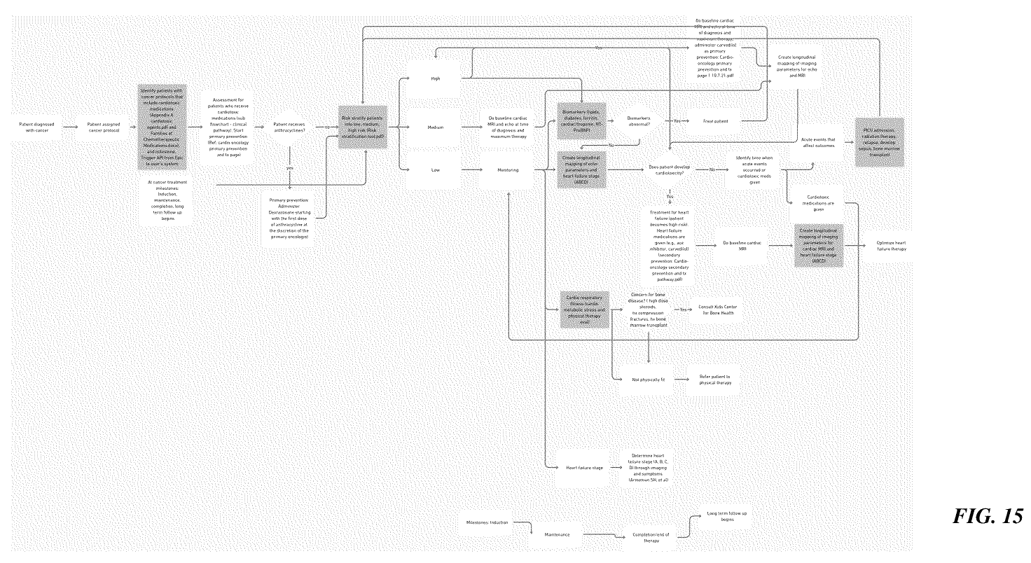

It should be appreciated that if the health care worker desires to have an MRI performed, one embodiment of an MRI algorithm that may be followed is shown in FIG. 13. FIG. 14A, FIG. 14B and FIG. 14C illustrates some embodiments of strategies for the administration of Dexrazoxane and Carvedilol and for Endocrinological evaluation, respectively. FIG. 15 is an operational flow diagram illustrating an embodiment of cardio-oncological treatment protocol.

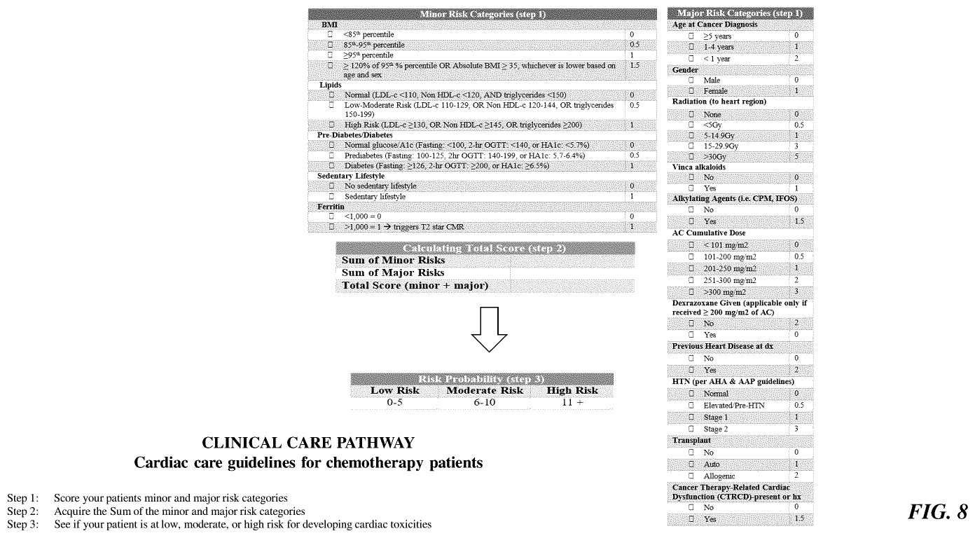

The method 500 further includes conducting a Risk Stratification (RS) evaluation on the patient, as shown in operational block 504. This RS evaluation may be conducted to assign a level of risk to the patient to determine if the patient is at a low risk, an intermediate risk or a high risk of experiencing cardiac issues (i.e., heart failure, heart disease, etc.). It should be appreciated that when performing the RS evaluation, the health care provider may use the RS tool shown in FIG. 8 to determine the risk level of the patient, wherein the health care worker may develop a score for the patient for minor and major risk categories. When scoring the patient for minor risk categories, the health care worker may evaluate the patient based on the following categories, 1) BMI, 2) Lipids, 3) Pre-Diabetes/Diabetes, 4) whether the patient has a sedentary lifestyle, and/or 5) the patient's ferritin level. For each of these categories, the health care worker may score the patient based on their evaluation of the patient.

When scoring the patient for major risk categories, the health care worker may evaluate the patient on the following categories, 1) the age of the patient at the time of cancer diagnosis, 2) the patient's gender, 3) whether the patient has received radiation to their heart region, 4) whether the patient has Vinca alkaloids, 5) whether the patient has Alkylating Agents (i.e., CPM, IFOS, etc.), 6) whether the patient has received an AC cumulative dose, 7) whether the patient has received Dexrazoxane, 8) whether the patient has been previously diagnosed with heart disease, 8) whether the patient has hypertension (per AHA & AAP guidelines), 9) whether the patient has received a transplant, and/or 10) whether the patient has Cancer Therapy-Related Cardiac Dysfunction (CTRCD) currently or in their history, whether the patient has received any potential cardiotoxic medications as presented in an embodiment shown in FIG. 11C. Once the health care provider has totaled the scores for the patient for the minor and major categories, the total score will identify the risk probability of the patient as being low risk, moderate (intermediate) risk or high risk experiencing cardiac complications.

It should be appreciated that the assessment shown in operational block 502 and the risk stratification shown in operational block 504 may be conducted at multiple periods during the treatment regime of the patient's care, such as at diagnosis, at maximal anthracycline therapy and/or at cancer therapy completion. It should also be appreciated that the patient may require evaluation of cardiac risk factors by a cardiologist and/or an oncologist at time of diagnosis to inform primary, secondary and tertiary prevention strategies. Furthermore, throughout therapy, the patient may require continual re-evaluation of risk factors. It should be appreciated that other time periods where re-evaluation of risk may be beneficial may include 1) at PICU admission, 2) when a relapse, refractory and/or a new cancer diagnosis occurs, 3) at radiation therapy, and/or 4) if a bone marrow transplant occurs.

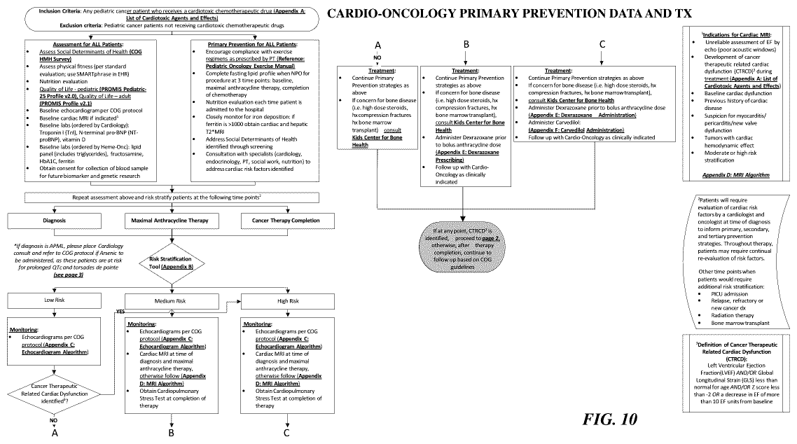

Once the RS has been determined, a treatment plan may be developed and personalized specifically for the patient, as shown in operational block 506. It should be appreciated that the treatment plan may vary responsive to the patient's risk level. Referring again to FIGS. 10-14C, an embodiment of a decision flow diagram and treatment protocols is shown to aid the Health Care Provider (HCP) in developing the patient's treatment plan. As shown, if the patient has a low risk of experiencing cardiac issues, the HCP may monitor the patient via echocardiograms per COG protocols and the HCP should identify if the patient has CTRCD. If the patient does have CTRCD, then the patient's risk level should be increased from low risk to high risk and the treatment plan should be developed based on the basis that the patient has a high risk of experiencing cardiac issues. If the patient does not have CTRCD, then the HCP should continue the primary prevention strategies developed earlier. If the HCP has a concern that the patient may experience bone disease (i.e., high dose steroids, hx compression fractures, hx bone marrow transplant, etc.) then the HCP may contact a specialist in bone health, such as Kids Center for Bone Health.

If the patient has an intermediate (medium) risk of cardiac issues, then the HCP may monitor the patient via echocardiograms per COG protocols and may conduct a cardiac MRI. The HCP may also obtain a cardiopulmonary stress test at the completion of the therapy. The treatment plan for a patient with an intermediate (medium) risk of cardiac issues may include continuation of the primary prevention strategies developed earlier. Again, if the HCP has a concern that the patient may experience bone disease (i.e., high dose steroids, hx compression fractures, hx bone marrow transplant, etc.) then the HCP may contact a specialist in bone health, such as Kids Center for Bone Health. The HCP may also administer Dexrazoxane prior to a bolus anthracycline dose and follow up with cardio-oncology as clinically indicated. If the patient has an high risk of cardiac issues, then the HCP may monitor the patient via echocardiograms per COG protocols and may conduct a cardiac MRI. The HCP may also obtain a cardiopulmonary stress test at the completion of the therapy. The treatment plan for a patient with an intermediate (medium) risk of cardiac issues may include continuation of the primary prevention strategies developed earlier. Again, if the HCP has a concern that the patient may experience bone disease (i.e., high dose steroids, hx compression fractures, hx bone marrow transplant, etc.) then the HCP may contact a specialist in bone health, such as Kids Center for Bone Health. The HCP may also administer Dexrazoxane prior to a bolus anthracycline dose and administer Carvedilol. The HCP may also follow up with cardio-oncology as clinically indicated. After therapy has been completed, the HCP should continue follow up based on COG guidelines. However, if at any point CTRCD is identified, then re-assessment should be conducted, as shown in operational block 508. According to one embodiment, CTRCD (and/or a change in systolic performance) may be defined as Left Ventricular Ejection Fraction (LVEF) and/or Global Longitudinal Strain (GLS) less than normal for age and/or Z score less than −2 or a decrease in Ejection Fraction (EF) of more than 10 EF units from baseline. If CTRCD is identified and/or if a patient develops a change in systolic performance during or after termination of cardiotoxic chemotherapy then the patient should be assessed by obtaining labs, such as troponin I (Tnl), N-terminal pro-BNP (NT-proBNP), vitamin D, lipid panel (may include triglycerides), fructosamine, HbA1C, ferritin, Chem 7, CBC, etc. Moreover, the HCP may also obtain a follow up cardiac MRI if the patient is stable for the procedure.

Responsive to the above, patient specific treatment may be developed and may include prescribing Enalapril or Lisinopril as shown in Table 1 below:

| | |
|---|---|
| 0-5 years of age: | Enalapril: 0.1 mg/kg/day BID, |
| | Titrate gradually over a week to a |
| | max dose of 0.3 mg/kg/day. |
| >5 years of age: | Enalapril 2.5 mg PO twice daily, |
| | Titrate gradually over a week to a |
| | max dose of 5 mg PO twice daily. |
| 12 years of age: | Lisinopril 2.5 mg/day. Titrate |
| | gradually over 1-2 weeks to a max |
| | dose of 10 mg PO once daily as |
| | tolerated per BP. |

Once ACE dose is maximized, add Carvedilol

Additionally, the HCP should continue primary prevention strategies, such as 1) encourage compliance with exercise regimens as prescribed by PT, 2) complete fasting lipid profile when NPO for procedure at 3 time points (baseline, maximal anthracycline therapy, completion of cancer therapy), 3) nutrition evaluation each time the patient is admitted to the hospital, 4) closely monitor for iron deposition and if ferritin is >1000 ng/ml, obtain cardiac and hepatic T2*MRI, 5) address SDOH identified through screening, and 6) consultation with specialists (cardiology, endocrinology, PT, social work, nutrition, etc.) to address cardiac risk factors identified.

As discussed above, the invention includes a system that may include (or communicate with) a cloud-based data repository which may be configured to receive data collected from a plurality of patients, wherein the data may be used to create an Artificial Intelligence (AI)/Machine Language (ML) model that will predict cardiotoxicity and its associated outcomes. One of the biggest disadvantages of current methods is that the data obtained from a plurality of patients is not being shared, analyzed and considered as a whole. Rather, the data is being analyzed locally (i.e., for each patient) and the benefits of a collective data repository is being lost. In an embodiment, the invention may allow for the collection of patient data for a plurality of patients to be continuously shared and processed via one or more algorithms resident in or communicated with one or more data repositories 104. This shared patient data may be applied and/or reapplied to AI/ML algorithms at various points during a patient's treatment protocol to continuously train and/or validate the AI/ML models. These AI/ML models may be used to predict the risk of cardiotoxicity and/or cardiotoxicity associated outcomes (i.e., heart failure, heart disease, etc.) for a patient throughout the patient's treatment.

In developing the overall AI/ML model(s), the invention may utilize one or more Machine Learning (ML) algorithms and Artificial Intelligence (AI) algorithms to assist in assessing the patient risk of developing cardiotoxicity, developing intervention and/or intervention strategies to achieve an optimal outcome and to predict the chances that heart failure and/or other cardiac issues may occur. The ML and/or AI models may systematically test include at least of the k-nearest neighbor (KNN) algorithm (and/or a modified version of the KNN algorithm), the support vector machine (SVM) algorithm (and/or a modified version of the SVM algorithm) and/or other ensemble methods (such as, random forest (RF) algorithms) to determine the importance of each of the factors pertaining to its prediction of the outcome of developing cardiotoxicity. The invention may further use one or more statistical cross-validation methods (i.e., k-fold cross-validation, holdout validation, stratified k-fold cross-validation, Leave-P-Out cross-validation) and nested cross-validation to estimate the performance or accuracy of the AI and/or ML model and to estimate the generalization error of the underlying model and to determine which hyperparameter need to be optimized. In an embodiment, variables that may be used in the feature set may include one or more of patient demographics, diagnosis, medications, imaging data, social determinants of health data, as well as data collected through PDAs (smartphone apps, etc.), genetic data, etc.

In an embodiment, the invention may use Reinforcement Learning (RL), such as the Markov Decision Process (MDP), to train the model to learn from past interventions and past outcomes to suggest a “best” intervention, given the current state of the patient. Variable sets that may be used in the training of this model may include one or more of the health state of the patient(s), intervention(s) that have occurred, transition probabilities that the patient may transition from one health state to the next health state given a certain intervention, past outcomes and the cost (or reward) associated with each intervention. It should be appreciated that in order to quantify the progression of heart failure based on phenotypes of patients and predict when and/or if heart failure may occur to inform interventions, use of statistical procedures such as survival analysis and/or random forest may be used. Variables which may be used with such analysis may include patient demographics, diagnosis, medications, imaging data, social determinants of health data, as well as data collected through PDAs (smartphone apps, etc.), genetic data, etc.

In an embodiment the system and method for training an Artificial Intelligence (AI) based decision-support system to predict cardiac outcomes in patients being treated with a cardiotoxic pharmaceutical is provided, includes processing the training dataset to generate an AI model to predict a probability of the patient experiencing a cardiac event, and processing the AI model using the validation dataset to generate an AI Model Accuracy (AIMA) value. If the AIMA value is less than an AIMA threshold value, the processing device may be configured to modify the AI model and reprocess the AI model using the validation dataset to redetermine the AIMA value. However, if the AIMA value is greater than the AIMA threshold value, the processing device may be configured to test the AI model with predetermined prospective data to generate an AI model performance value. If the AI model performance value is less than an AI threshold value, the processing device may be configured to reprocess the AI model using the patient data using at least one of the AI algorithm and the ML algorithm. However, if the AI model performance value is greater than the AI threshold value, the processing device may be configured to implement the AI model. It should be appreciated that the AIMA threshold value and/or the AI threshold value may be any threshold valued desired and/or suitable to the desired end purpose. For example, in one embodiment, the AIMA threshold value may be 80%, while in another embodiment, the AIMA threshold value may be 90%. Also, in one embodiment, the AI threshold value may be 75%, while in another embodiment, the AI threshold value may be 90%. It is contemplated that the AIMA threshold value and the AI threshold value may change and become more (or less) accurate during each iteration.

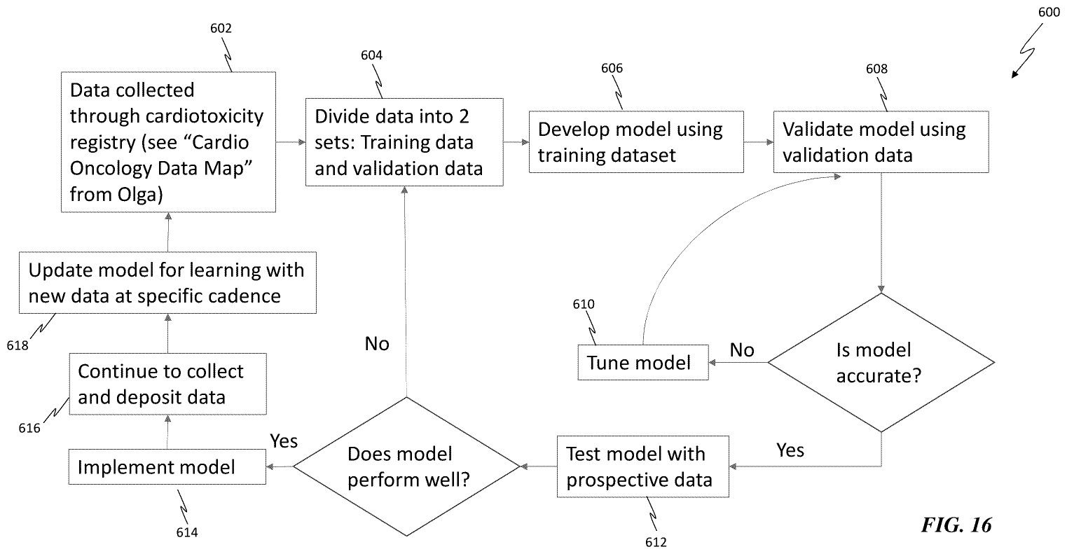

Referring to FIG. 16, a method 600 for predicting cardiotoxicity using the Intelligent Recommendation Engine (IRE) 100 is provided, in accordance with an embodiment. The method 600 includes collecting patient data, as shown in operational block 602, wherein the patient data may be collected through the cardiotoxicity registry, health care providers, and/or one or more DPIDs 106. The method 500 further includes dividing the patient data into two (2) or more datasets where one dataset may be used as a training dataset and the other dataset may be used as validation data, as shown in operational block 604. The training dataset may be used to develop the prediction model, as shown in operational block 606, and the validation dataset may be used to validate the prediction model to ensure that the model is accurate, as shown in operational block 608. It the model is not accurate, then the model may be modified to account for the inaccuracies, as shown in operational block 610. However, if the model is accurate, then the model is tested with prospective data to determine how well the model performs, as shown in operational block 612. If the model does not perform well, then the dataset is divided again and used for retraining and revalidating. If the model does perform well, then the model is implemented, as shown in operational block 614 and data collection continues with the collected data being introduced into the DP 102, as shown in operational block 616. The model is updated for learning at a specific cadence with the newly collected data being introduced in the DR 104, as shown in operational block 618.

It should be appreciated that the method 600 may be modified as desired and/or based on unanticipated challenges with data collection and data processing. One unique and novel feature of the invention is the use of these models with the technology and the clinical insight to optimize the workflow for providing intervention with speed and precision to the cardiotoxic population.

While the invention has been described with reference to an exemplary embodiment, it should be understood by those skilled in the art that various changes may be made and equivalents may be substituted for elements thereof without departing from the scope of the invention. Moreover, the embodiments or parts of the embodiments may be combined in whole or in part without departing from the scope of the invention. In addition, many modifications may be made to adapt a particular situation or material to the teachings of the invention without departing from the scope thereof. Therefore, it is intended that the invention not be limited to the particular embodiment disclosed as the best mode contemplated for carrying out this invention, but that the invention will include all embodiments falling within the scope of the appended claims. Moreover, unless specifically stated any use of the terms first, second, etc. do not denote any order or importance, but rather the terms first, second, etc. are used to distinguish one element from another.

What is claimed is:

1. An Artificial Intelligence (AI) based decision-support system for providing personalized cardio-oncology care to a patient being treated with a cardiotoxic pharmaceutical, the system comprising:
a Data Pipeline (DP);
a Data Repository (DR); and
a processing device associated with the DR,
wherein the DP is configured to receive patient data and communicate the patient data to the DR, and
wherein the DR is configured to receive the patient data from the DP and communicate the patient data to the processing device,
wherein the processing device is configured to execute at least one of an Artificial Intelligence Algorithm (AIA) and a Machine Learning Algorithm (MLA) to divide the patient data into a training dataset and a validation dataset,
wherein at least one of the AIA and the MLA are configured to process the training dataset to generate an AI model to predict a probability of the patient experiencing a cardiac event, and
wherein at least one of the AIA and the MLA are configured to process the AI model using the validation dataset to generate an AI Model Accuracy (AIMA) value.

2. The system of claim 1, wherein the DP is configured to receive the patient data from a Data Pipeline Input Device (DPIP).

3. The system of claim 1, wherein the Data Pipeline (DP) includes at least one of a DP computer server and a DP processing device, wherein the DP computer server and DP processing device are configured to communicate the patient data to the DR via at least one of a hardwired communication and a wireless communication.

4. The system of claim 2, wherein the DPIP includes at least one of an ECG/EKG machine, a blood pressure machine, a heart rate monitor, an MRI machine, a CT Scanner, an ultrasound machine, a computer, a laptop, a tablet, a smartphone, a PDA and a Patient Records Registry.

5. The system of claim 1, wherein,
if the AIMA value is less than an AIMA threshold value, the processing device is configured to modify the AI model and reprocess the AI model using the validation dataset to redetermine the AIMA value; and
if the AIMA value is greater than the AIMA threshold value, the processing device is configured to test the AI model with predetermined prospective data to generate an AI model performance value.

6. The system of claim 5, wherein,
if the AI model performance value is less than an AI threshold value, the processing device is configured to reprocess the AI model using the patient data using at least one of the AI algorithm and the ML algorithm; and
if the AI model performance value is greater than the AI threshold value, the processing device is configured to implement the AI model.

7. The system of claim 6, wherein
if the AI model is implemented, the processor is configured to,
collect new patient data;
communicate the new patient data with the DR; and
process the AI model using the new patient data to update the AI model; and
communicate the AI model and the new patient data to the DR.

8. The system of claim 1, wherein the cardiac event is at least one of heart failure, heart disease and myocardial infarction.

9. An Artificial Intelligence (AI) based decision-support system for providing personalized cardio-oncology care to a patient being treated with a cardiotoxic pharmaceutical, the system comprising:
a Data Pipeline (DP);
a Data Repository (DR); and
a processing device associated with the DR,
wherein the DP is configured to receive patient data and communicate the patient data to the DR, and
wherein the DR is configured to receive the patient data from the DP and communicate the patient data to the processing device,
wherein the processing device is configured to execute at least one of an Artificial Intelligence Algorithm (AIA) and a Machine Learning Algorithm (MLA) to process at least a portion of the patient data to generate an AI model to predict a probability of the patient experiencing a cardiac event and to generate and AI Model Accuracy (AIMA) value.

10. The system of claim 9, wherein the processing device is configured to divide the patient data into a training dataset and a validation dataset,
wherein at least one of the AIA and the MLA are configured to process the training dataset to generate the AI model, and
wherein at least one of the AIA and the MLA are configured to process the AI model using the validation dataset to generate the AI Model Accuracy (AIMA) value.

11. The system of claim 9, wherein the DP is configured to receive the patient data from a Data Pipeline Input Device (DPIP).

12. The system of claim 9, wherein the Data Pipeline (DP) includes at least one of a DP computer server and a DP processing device, wherein the DP computer server and DP processing device are configured to communicate the patient data to the DR via at least one of a hardwired communication and a wireless communication.

13. The system of claim 11, wherein the DPIP includes at least one of an ECG/EKG machine, a blood pressure machine, a heart rate monitor, an MRI machine, a CT Scanner, an ultrasound machine, a computer, a laptop, a tablet, a smartphone, a PDA and a Patient Records Registry.

14. The system of claim 9, wherein,
if the AIMA value is less than an AIMA threshold value, the processing device is configured to modify the AI model and reprocess the AI model using the validation dataset to redetermine the AIMA value; and
if the AIMA value is greater than the AIMA threshold value, the processing device is configured to test the AI model with predetermined prospective data to generate an AI model performance value.

15. The system of claim 14, wherein,
if the AI model performance value is less than an AI threshold value, the processing device is configured to reprocess the AI model using the patient data using at least one of the AI algorithm and the ML algorithm; and
if the AI model performance value is greater than the AI threshold value, the processing device is configured to implement the AI model.

16. The system of claim 15, wherein
if the AI model is implemented, the processor is configured to,
collect new patient data;
communicate the new patient data with the DR; and
process the AI model using the new patient data to update the AI model; and
communicate the AI model and the new patient data to the DR.

17. The system of claim 9, wherein the cardiac event is at least one of heart failure, heart disease and myocardial infarction.

18. A method for training an Artificial Intelligence (AI) based decision-support system to predict cardiac issues in patients being treated with a cardiotoxic pharmaceutical, wherein the system includes a Data Pipeline (DP) communicated with a Data Repository (DR) and a processing device associated with the data repository, the method comprising:
receiving patient data via the DP;
communicating the patient data to the DR and the processing device; and
processing the patient data to execute at least one of an Artificial Intelligence Algorithm (AIA) and a Machine Learning Algorithm (MLA) to divide the patient data into a training dataset and a validation dataset,
processing the training dataset to generate an AI model to predict a probability of the patient experiencing a cardiac event, and
processing the AI model using the validation dataset to generate an AI Model Accuracy (AIMA) value.

19. The method of claim 18, further comprising,
if the AIMA value is less than an AIMA threshold value,
modify the AI model, and
reprocessing the AI model using the validation dataset to redetermine the AIMA value; and
if the AIMA value is greater than the AIMA threshold value,
testing the AI model with predetermined prospective data to generate an AI model performance value, wherein
if the AI model performance value is less than an AI threshold value, reprocessing the AI model using the patient data; and
if the AI model performance value is greater than the AI threshold value, implementing the AI model.

20. The method of claim 19, further comprising,
if the AI model is implemented,
collecting new patient data;
communicating the new patient data with the DR; and
processing the AI model using the new patient data to update the AI model; and
communicating the AI model and the new patient data to the DR.
